(19) United States
(12) Patent Application Publication
(10) Pub. No.: US 2007/0249978 A1
(43) Pub. Date: Oct. 25, 2007

(54) Weighted belt

(75) Inventor: Craig E. Curphey, Barrie (CA)

Correspondence Address: PEARNE & GORDON LLP, 1801 EAST 9TH STREET, SUITE 1200, CLEVELAND, OH, 44114-3108, US

(73) Assignee: Wellness Belts Inc.

(21) Appl. No.: 11/475,310

(22) Filed: Jun. 27, 2006

(30) Foreign Application Priority Data
Apr. 20, 2006 (CA) 2,544,229

(51) Int. Cl. A61F 5/00

(52) U.S. Cl. 602/19

(57) ABSTRACT

A weighted belt is provided. The weighted belt comprises a belt body that defines a body pocket; a plurality of weights disposed within the body pocket; a first cinching end that extends from an end of the belt body; and, a second cinching end that extends from an opposite end of the belt body. The first and second cinching ends comprise a suitable dress-belt material disposed on at least a portion of the surface of the belt body sufficient to conceal at least partially the belt body when worn and viewed face on, and are configured for releasable engagement.

### FIELD OF INVENTION

The present invention relates to belts, in general. In particular, the present invention relates to weighted belts.

### BACKGROUND OF THE INVENTION

People need exercise. Weight bearing exercise has been shown to increase bone mass and thereby prevent or delay the onset of degenerative disorders such as osteoporosis. However, increased sedentary lifestyles leave little time or opportunity for such exercise. There exists a need for providing weight bearing exercise that accommodates increasingly sedentary lifestyles.

### SUMMARY OF THE INVENTION

A weighted belt is provided. The weighted belt comprises a belt body that defines a body pocket; a plurality of weights disposed within the body pocket; a first cinching end that extends from an end of the belt body; and, a second cinching end that extends from an opposite end of the belt body. The first and second cinching ends comprise a suitable dress-belt material disposed on at least a portion of the surface of the belt body sufficient to conceal the belt body when worn and viewed face on, and are configured for releasable engagement.

The belt body may have a length sufficient to span a belt wearer's back and at least a portion of the belt wearer's sides.

The belt body may have a length sufficient to span a belt wearer's back, sides and at least a portion of the belt wearer's front.

Each of the plurality weights may articulate with an adjacent weight in at least two axes of movement.

Each of the plurality of weights may comprise a lead alloy.

The lead alloy may comprise lead and antimony.

### LIST OF FIGURES

FIG. 1 is a cut-away perspective view of a belt according to an embodiment of the present invention;

FIG. 2 is plan view of an end of the belt of FIG. 1;

FIG. 3 is a cut-away plan view of a portion of the belt of FIG. 1;

FIG. 4 is a cross-sectional view the plurality of weights of the belt of FIG. 1;

FIG. 5a is a cross-sectional view illustrating relative movement of adjacent weights of the belt of FIG. 1;

FIG. 5b is a plan view illustrating relative movement of adjacent weights of the belt of FIG. 1; and,

FIG. 6 is a transverse cross-sectional view illustrating an end of the belt of FIG. 1.

### DETAILED DESCRIPTION

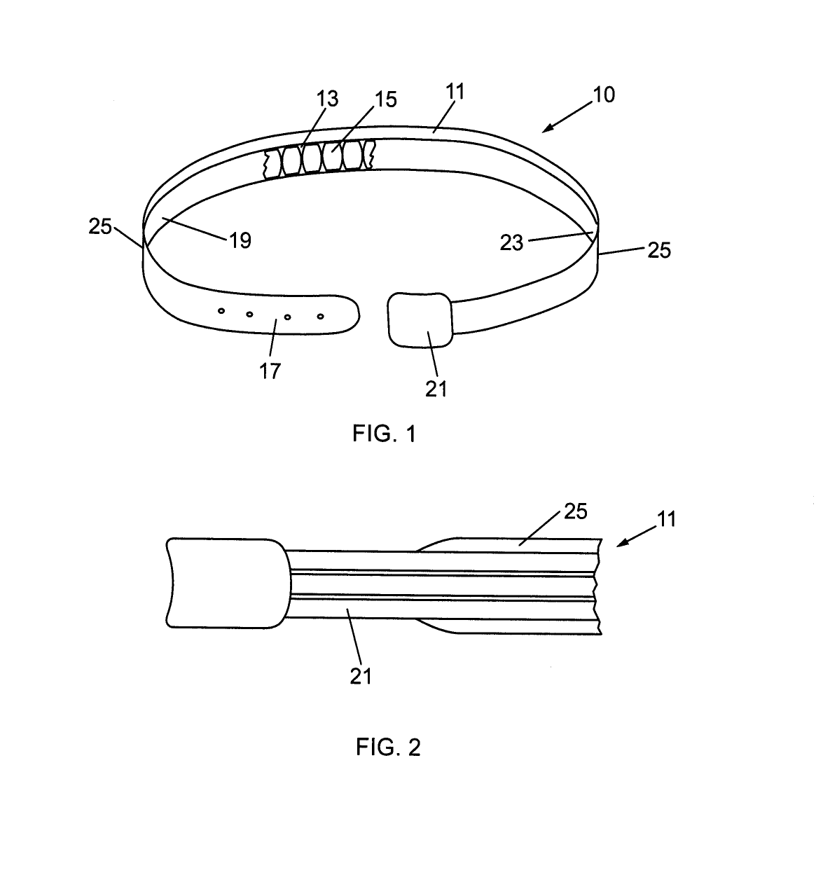

Referring to FIG. 1, a weighted belt 10 is illustrated according to an embodiment of the present invention. The weighted belt 10 comprises a belt body 11 that defines a body pocket 13; a plurality of weights 15 disposed within the body pocket 13; a first cinching end 17 that extends from an end 19 of the belt body 11; and, a second cinching end 21 that extends from an opposite end 23 of the belt body 11. The first and second cinching ends (17, 21) comprise a suitable dress-belt material disposed on at least a portion of the surface 25 of the belt body 11 sufficient to conceal at least partially the belt body 11 when worn and viewed face on, and are configured for releasable engagement.

In a preferred embodiment, the belt body 11 has a length sufficient to span a belt wearer's back and at least a portion of the belt wearer's sides. However, it will be apparent to those skilled in the art that the selected length of the belt body 11 may be determined by the wearer's needs. For example, the belt body 11 may have a length sufficient to span a belt wearer's back, sides and at least a portion of the belt wearer's front.

The body pocket 13, which preferably runs the length of the belt body 11, may be sized as required.

The dress-belt material may be leather or a material made to resemble leather. Any material that is made to resemble a dress-belt material may be employed, such as various natural or synthetic fabrics. It will be understood that the particular material selected may be determined by the wearer's dress style. For example, if a business look is required, a material appropriate for a business environment would be selected. If the belt is to be worn in a less formal environment, a belt incorporating the appropriate material may be selected. The cinching ends 17, 21 may be connected to the belt body 11 by any means known to those skilled in the art. For example, they may be stitched, stapled, riveted or adhered to the belt body 11.

Referring to FIG. 2, the cinching end 21 of the weighted belt 10 is illustrated. The cinching end 21 extends from end 23 of the belt body 11. FIG. 2 also illustrates that the end 21 is disposed on at least a portion of the surface 25 sufficient to conceal at least partially the belt body 11 when the belt 10 is worn and viewed face on. The cinching ends 17, 21 serve at least two functions; the first being to secure releasably the belt 10 on the wearer's waist; the second being to conceal at least the portion of the belt body 11 that is viewable when the belt 10 is worn. In an alternate embodiment, the cinching ends 17, 21 completely conceal the belt body 11.

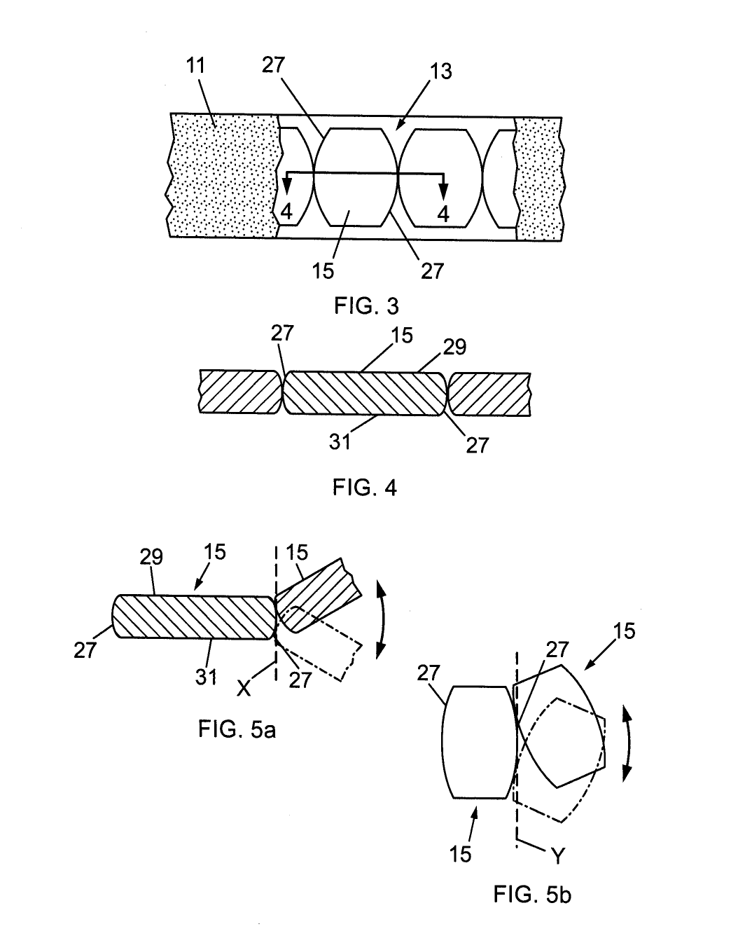

Referring to FIG. 3, a cut-away portion of the belt body 11 is illustrated. The cut-away exposes the plurality of weights 15, which are aligned side-by-side when the weights 15 are disposed within the belt body 11. Each weight 15 preferably has curved sides (or face) 27, each of which providing an articulating surface for interacting with a curved side 27 of an adjacent weight 15. The curved sides 27 give each weight 15 a generally flattened or truncated ellipsoid shape in outline (as is illustrated in FIGS. 3 and 5(b)).

Referring to FIG. 4, a transverse cross-sectional profile of the weights 15 of FIG. 3 are illustrated. Each of the weights 15 has opposing inside and outside faces (29, 31), in addition to the opposing side faces 27. Preferably, the inside and outside faces (29, 31) are generally parallel to each other. While the faces 29, 31 may define a flat or uncurved surface, they may be slightly curved. For example, the inside face 29 may be slightly cupped (or concave) over its length and the outside face 31 may define a complementary bowed (or convex) surface. The cupped inside face 29 would be positioned adjacent the body when the weighted belt 10 is in use. It will be apparent to those skilled in the art that the inside face 29 need not be cupped and that the outside face 31 need not have a complementary bowed surface. The size of the weights 15 and the degree of curving may be varied to suit the particular user's weighting requirements.

Still referring to FIG. 4 and referring to FIG. 5(a), the opposing side faces 27 of weight 15 are illustrated. The side faces are preferably rounded when viewed in transverse cross-section, thereby providing an articulating surface to engage the side face 27 of an adjacent weight 15. The degree of curvature provided may be varied according to the range of articulating motion required. For example, a 180° of curvature may be used if a belt having a relatively short radius of curvature were required, while a smaller degree of side 27 curvature may be used to provide a belt with a longer radius of curvature. In each case, however, the curved face 27 provides an articulating surface, which permits the weighted belt 10 to more closely hug the waist of a user. The curved face 27 may also permit closer alignment of the weights 15 compared to weights not having curved faces 27.

Referring to FIG. 3 and referring to FIG. 5(b), the opposing side faces 27 of weight 15 are illustrated. The side faces are preferably rounded when viewed in longitudinal cross-section, thereby providing an articulating surface to engage the side face 27 of an adjacent weight 15. The degree of curvature provided may be varied according to the range of articulating motion required. The curved face 27 provides an articulating surface, which permits, among other things, the wearer of the weighted belt 10 to more easily bend at the waist.

Referring to FIGS. 5(a) and 5(b), the weights 15 are illustrated in relation to each other. The curved faces 27 provide articulating surfaces along respective axes of movement (X,Y). Preferably, each of the plurality weights 15 articulates with an adjacent weight 15 in at least the two axes of movement (X,Y).

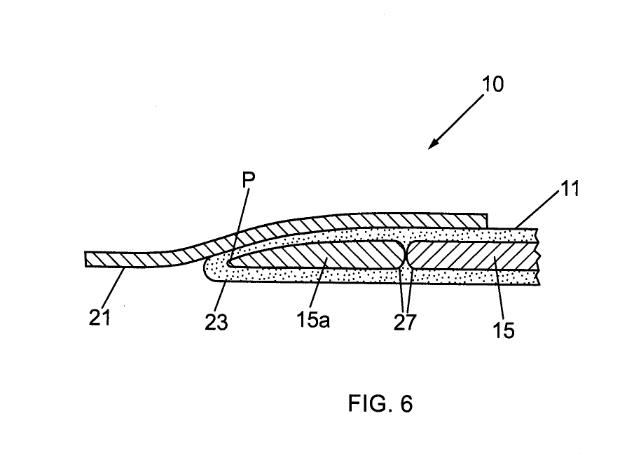

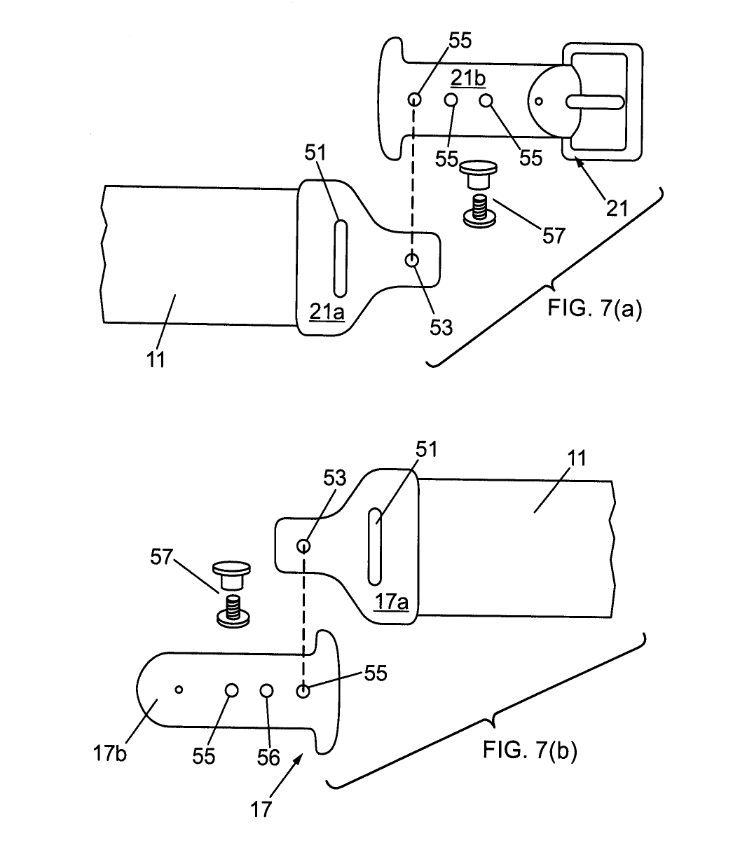

Referring to FIG. 6, a transverse cross-sectional view of an end of the belt 10 is illustrated. A terminal weight 15a is illustrated in the belt's 10 assembled position. Unlike the shape of weight 15, the terminal weight 15a is preferably tapered at a terminal point P. In the transverse cross-sectional view, the terminal weight 15a is almost bullet shaped. This particular shape allows for a smoother transition from the belt body end (e.g., 23) to the cinching end (e.g., 21) than would otherwise be the case. It will be apparent to those skilled in the art that any shape or configuration that provides a smoother transition may be employed.

In a preferred embodiment, each of the plurality of weights comprises a lead alloy, such as lead and antimony. The lead alloy comprises about 97% lead and about 3% antimony. The lead alloy preferably comprises at least 96% lead and at least 2.9% antimony. More particularly, the lead alloy comprises from 96.7% to 97.1% lead and from 2.9% to 3.3% antimony. The lead alloy may also contain other elements in trace amounts, such as nickel, silver and copper (see table below). The respective amounts of the these trace elements may vary with factors such as the source of the raw material and the efficiency of the alloy production process. The non-lead elemental composition of a sample batch of weight 15 is provided in the table below.

Weight 15 Elemental Analysis:

| Element | Specification Limit | Analysis (%) |
|---|---|---|
| Sb | 2.90 and 3.25 | 3.18 |
| Sn | 0.15 and 0.25 | 0.21 |
| As | 0.15 and | 0.22 |
| Cu | less than 0.04 | <0.001 |
| Bi | less than 0.05 | 0.01 |
| Ag | less than 0.10 | 0.03 |
| Ni | less than 0.001 | <0.001 |
| S | less than 0.001 | <0.001 |

The above description is intended in an illustrative rather than restrictive sense. Variations may be apparent to those skilled in the art without departing from the spirit and scope of the invention as defined by the claims set out below.

I claim:

1. A weighted belt comprising:
a belt body defining a body pocket;
a plurality of weights disposed within the body pocket;
a first cinching end extending from an end of the belt body; and,
a second cinching end extending from an opposite end of the belt body, the first and second cinching ends:
comprising a suitable dress-belt material disposed on at least a portion of the surface of the belt body sufficient to conceal at least partially the belt body when worn and viewed face on, and
being configured for releasable engagement.

2. A weighted belt according to claim 1, wherein the belt body has a length sufficient to span a belt wearer's back and at least a portion of the belt wearer's sides.

3. A weighted belt according to claim 1, wherein the body pocket runs the length of the belt body.

4. A weighted belt according to claim 1, wherein the dress-belt material is leather.

5. A weighted belt according to claim 1, wherein the dress-belt material is a material made to resemble leather.

6. A weighted belt according to claim 1, wherein each of the plurality weights articulates with an adjacent weight in at least two axes of movement.

7. A weighted belt according to claim 1, wherein each of the plurality of weights is curved along an axis of movement.

8. A weighted belt according to claim 6, wherein each of the plurality of weights is truncated elliptical in outline.

9. A weighted belt according to claim 1, wherein each of the plurality of weights comprises a lead alloy.

10. A weighted belt according to claim 9, wherein the lead alloy comprises lead and antimony.

11. A weighted belt according to claim 10, wherein the lead alloy comprises at least 96% lead and at least 2.9% antimony.

12. A weighted belt according to claim 11, wherein the lead alloy comprises from 96.7% to 97.1% lead and from 2.9% to 3.3% antimony.

13. A weighted belt according to claim 10, wherein the lead alloy comprises about 97% lead and about 3% antimony.

14. A weighted belt comprising:
a belt body defining a body pocket, the belt body having a length sufficient to span a belt wearer's back and at least a portion of the belt wearer's sides;
a plurality of weights disposed within the body pocket, the weights being articulatable with respect to each other;
a first cinching end extending from an end of the belt body; and,
a second cinching end extending from an opposite end of the belt body, the first and second cinching ends:
comprising a suitable dress-belt material disposed on at least a portion of the surface of the belt body sufficient to conceal at least partially the belt body when worn and viewed face on, and
being configured for releasable engagement.

15. A weighted belt according to claim 14, wherein each of the plurality weights articulates with an adjacent weight in at least two axes of movement.

16. A weighted belt according to claim 15, wherein each of the plurality of weights comprises a lead alloy.

17. A weighted belt according to claim 16, wherein the lead alloy comprises lead and antimony.

18. A weighted belt according to claim 17, wherein the lead alloy comprises at least 96% lead and at least 2.9% antimony.

19. A weighted belt according to claim 17, wherein the lead alloy comprises about 97% lead and about 3% antimony.

20. A weighted belt according to claim 7, wherein each of the plurality of weights is truncated elliptical in outline.
